(19) United States
(12) Patent Application Publication
(10) Pub. No.: US 2025/0049730 A1
(43) Pub. Date: Feb. 13, 2025

(54) ULTRA SMALL GELATIN NANOPARTICLES, COMPOSITE STRUCTURES AND SYNTHESIS METHOD

(71) Applicant: THE CURATORS OF THE UNIVERSITY OF MISSOURI, COLUMBIA, MO (US)

(72) Inventors: Raghuraman Kannan, Columbia, MO (US); Dhananjay Suresh, Columbia, MO (US)

(21) Appl. No.: 18/721,126

(22) PCT Filed: Nov. 21, 2022
(86) PCT No.: PCT/US2022/050550

§ 371 Date: Jun. 17, 2024

Related U.S. Application Data
(60) Provisional application No. 63/282,846, filed on Nov. 24, 2021.

(51) Int. Cl. A61K 9/51; A61K 31/704; A61K 33/242; A61K 33/243; A61K 49/04

(52) U.S. Cl. CPC A61K 9/5169; A61K 31/704; A61K 33/242; A61K 33/243; A61K 49/0438

(57) ABSTRACT

A method for synthesizing ultrasmall gelatin nanoparticles. A first desolvation is conducted to produce gelatin strands in a first solution and then setting the pH of the solution to charge and separate the strands. Tripolyphosphate (TPP) is added to the first solution to form gelatin-TPP-gelatin bridges. Second desolvation of the gelatin-TPP-gelatin bridges is conducted in a second solution. The second solution contains an TPP-Ethanol:Acetone mixture in a ratio between 1:1 and 1:5 and TPP between 0.005-0.025 vol % or a Glutaraldehyde-Ethanol:Acetone mixture in a ratio of glutaraldehyde-ethanol:Acetone range of 2.5-12% v/v to produce a colloid of self-assembled gelatin nanoparticles. The method can produce nanomaterial consisting of a plurality of gelatin nanoparticles sized at ˜10 nm. The nanomaterial can encapsulate a drug, metal nanoparticle or contrast agent.

### PRIORITY CLAIM AND REFERENCE TO RELATED APPLICATION

The application claims priority under 35 U.S.C. § 119 and all applicable statutes and treaties from prior U.S. provisional application Ser. No. 63/282,846 which was filed Nov. 24, 2021.

### FIELD

A field of the invention is gelatin nanomaterials, including gelatin nanoparticles and composite materials including gelatin nanoparticles. Example applications of the invention include biomedical applications such as disease treatment and disease detection.

### BACKGROUND

Gelatin nanoparticles are useful, for example, to target tumors in the treatment of cancer. Gelatin nanoparticles sized at less than 50 nm are optimum for deep tumor penetration. Most known methods for synthesis of gelatin nanoparticles produce gelatin nanoparticles consisting or including a distribution of sizes larger than 50 nm, and the larger nanoparticles tend to accumulate on the surface of tumors instead of penetrating within the interior space of the tumor.

One method to synthesize gelatin nanoparticles is a two-step desolvation method disclosed in Kannan et al, U.S. Pat. No. 10,426,842. A gelatin solution is prepared and then desolvated via a first step with acetone, and a second step of stepwise addition of acetone, with crosslinking via glutaraldehyde. The method forms ˜200 nm gelatin nanoparticles. Kannan US Published Application Number 2018/0008551 also discloses gel nanoparticles of ˜220 nm in size.

Skardal and Clark US Published Application No. 20200108172 discloses gelatin nanoparticles having a diameter, on average, of about 150, 200, 250, or 300 nm to about 350, 400, 450, or 500 nm. The application also report cross-linking of the particles with glutaraldehyde and aggregation of the particles to sizes of 1-9 μm. The gelatin nanoparticles in the application are described as being formed according to Coester, C. J. et al., “Gelatin Nanoparticles By Two Step Desolvation A New Preparation Method, Surface Modifications And Cell Uptake,” Journal of Microencapsulation, vol 17, issue 2, pp. 187-193 (2000). That process used acetone for the first desolvation followed and acetone added dropwise in the second solution followed by addition of glutaraldehyde for cross-linking. The particles were sized at ˜270 nm.

Wong et al., US Published Application No. 20130224282 reports data including ˜100 nm gelatin nanoparticles encompassing 10 nm quantum dots. The gel nanoparticles were formed with two-step desolvation of acetone desolvation of an initial gel followed by acetone desolvation glutaraldehyde cross-linking. The particles are modified to easily dissolve in vivo and release encapsulated 10 nm payloads.

Srivastava et al. US Published patent Application No. 20110229580 reports data showing nanoparticles of 200 nm or larger.

Coester et al., “Gelatin nanoparticles by two-step desolvation-a new preparation method, surface modifications and cell uptake,” J.

Microencapsulation, 2000, Vol 17 No 2, 187-193 report 60 nm gelatin nanoparticles were generated using a two-step desolvation process of treatment with 1) acetone then 2) acetone and glutaraldehyde to crosslink. The crosslinking is performed after the second desolvation step.

Xuezhen Zhai reports, in a published dissertation “Gelatin nanoparticles & nanocrystals for dermal delivery https://d-ub.info/1054636958/34 (published dissertation), data showing gelatin nanoparticles of 50-100 nm through optimization of the standard acetone two-step desolvation process. While gelatin concentrations, incubation times, temperature, pH, and type of desolvating agent were experimented with, the methods followed the standard two-step desolvation process with glutaraldehyde used to crosslink at the end of the process.

Kaintura et al, “Gelatin nanoparticles as a delivery system for proteins,” J Nanomed Res 2 (1): 00018. DOI: 10.15406/jnmr.2015.02.00018, report data showing 100-350 nm sized gel nanoparticles. There are references to smaller particles without data support for the same, and no description of a method that supports generation of smaller particles as the method merely discusses dispersing gelatin in water and stirring followed by acetone treatment, which treatment in articles above as producing larger gel nanoparticles of about 200 nm, or the reported data in this article showing 100-350 nm sized gel nanoparticles.

Another example method is disclosed in Li et al, “Amphiphilically-modified gelatin nanoparticles: Self-assembly behavior, controlled biodegradability, and rapid cellular uptake for intracellular drug delivery,” J. Mater. Chem., 2011, 21, 12381 (formation of ˜60-130 nm gelatin nanoparticles using a synthesis method consisting of the hexanoic anhydride treatment of gelatin followed by self-assembly).

A published method by Naidu and Paulson, “A New Method for the Preparation of Gelatin Nanoparticles: Encapsulation and Drug Release Characteristics, Journal of Applied Polymer Science 121 (6): 3495-3500 reports the formation of ˜30-40 nm gel nanoparticles for use as carriers for drug release applications. The synthesis method describes crosslinking via glutaraledehyde prior to desolvation using between 8:2 and 9:1 methanol:water. The nanoparticles produced were in the range of ˜30-40 nm and swell to ˜110-130 nm when dispersed in water. Some control over the size distribution of gel nanoparticles via the water-methanol ratio, polymer concentration and GA content. Distributions are produced by the reported experiments, with the distributions including the smallest gel nanoparticles including 30 nm and larger particles. Some smaller particles appear to be shown in FIG. 2 of the article, but those can include impurities, trace particle formation, and there is no explanation of how to avoid having a distribution including larger particles. The article only demonstrates control over particle synthesis including a distribution of 30-40 nm particles.

### ADDITIONAL REFERENCES CONCERNING NANOMATERIALS

S. Azarmi, Y. Huang, H. Chen, S. McQuarrie, D. Abrams, W. Roa, W. H. Finlay, G. G. Miller, R. Löbenberg, Optimization of a two-step desolvation method for preparing gelatin nanoparticles and cell uptake studies in 143B osteosarcoma cancer cells, J Pharm Pharmaceut Sci, 9 (2006) 124-132.
S. M. Ahsan, C. M. Rao, The role of surface charge in the desolvation process of gelatin: implications in nanoparticle synthesis and modulation of drug release, International Journal of Nanomedicine, Volume 12 (2017) 795-808.
M. Papi, V. Palmieri, G. Maulucci, G. Arcovito, E. Greco, G. Quintiliani, M. Fraziano, M. De Spirito, Controlled self assembly of collagen nanoparticle, Journal of Nanoparticle Research, 13 (2011) 6141-6147.
P. C. Bessa, R. Machado, S. Nürnberger, D. Dopler, A. Banerjee, A. M. Cunha, J. C. Rodríguez-Cabello, H. Redl, M. van Griensven, R. L. Reis, M. Casal, Thermoresponsive self-assembled elastin-based nanoparticles for delivery of BMPs, Journal of Controlled Release, 142 (2010) 312-318.
A. Kimura, J.-i. Jo, F. Yoshida, Z. Hong, Y. Tabata, A. Sumiyoshi, M. Taguchi, I. Aoki, Ultra-small size gelatin nanogel as a blood brain barrier impermeable contrast agent for magnetic resonance imaging, Acta Biomaterialia, 125 (2021) 290-299.
A. M. Etorki, M. Gao, R. Sadeghi, L. F. Maldonado-Mejia, J. L. Kokini, Effects of Desolvating Agent Types, Ratios, and Temperature on Size and Nanostructure of Nanoparticles from a-Lactalbumin and Ovalbumin, Journal of Food Science, 81 (2016).
P. Khramtsov, O. Burdina, S. Lazarev, A. Novokshonova, M. Bochkova, V. Timganova, D. Kiselkov, A. Minin, S. Zamorina, M. Rayev, Modified Desolvation Method Enables Simple One-Step Synthesis of Gelatin Nanoparticles from Different Gelatin Types with Any Bloom Values, Pharmaceutics, 13 (2021).
A. O. Elzoghby, Gelatin-based nanoparticles as drug and gene delivery systems: Reviewing three decades of research, Journal of Controlled Release, 172 (2013);
M. J. Sailor, J.-H. Park, Hybrid Nanoparticles for Detection and Treatment of Cancer, Advanced Materials, 24 (2012) 3779-3802.
M. Ha, J.-H. Kim, M. You, Q. Li, C. Fan, J.-M. Nam, Multicomponent Plasmonic Nanoparticles: From Heterostructured Nanoparticles to Colloidal Composite Nanostructures, Chemical Reviews, 119 (2019);
N. P. B. Tan, C. H. Lee, Environment-Friendly Approach in the Synthesis of Metal/Polymeric Nanocomposite Particles and Their Catalytic Activities on the Reduction of p-Nitrophenol to p-Aminophenol, Green Chemical Processing and Synthesis 2017.
B. Gong, Y. Shen, H. Li, X. Li, X. Huan, J. Zhou, Y. Chen, J. Wu, W. Li, Thermo-responsive polymer encapsulated gold nanorods for single continuous wave laser-induced photodynamic/photothermal tumour therapy, Journal of Nanobiotechnology, 19 (2021).
G. Yang, Z. Xiao, H. Long, K. Ma, J. Zhang, X. Ren, J. Zhang, Assessment of the characteristics and biocompatibility of gelatin sponge scaffolds prepared by various crosslinking methods, Scientific Reports, 8 (2018).
S. Amjadi, H. Hamishehkar, M. Ghorbani, A novel smart PEGylated gelatin nanoparticle for co-delivery of doxorubicin and betanin: A strategy for enhancing the therapeutic efficacy of chemotherapy, Materials Science and Engineering: C, 97 (2019) 833-841.
S. K. Jain, Y. Gupta, A. Jain, A. R. Saxena, P. Khare, A. Jain, Mannosylated gelatin nanoparticles bearing an anti-HIV drug didanosine for site-specific delivery, Nanomedicine: Nanotechnology, Biology and Medicine, 4 (2008).
E. J. Lee, S. A. Khan, J. K. Park, K.-H. Lim, Studies on the characteristics of drug-loaded gelatin nanoparticles prepared by nanoprecipitation, Bioprocess and Biosystems Engineering, 35 (2011) 297-307.
C. Kuttner, R. P. M. Höller, M. Quintanilla, M. J. Schnepf, M. Dulle, A. Fery, L. M. Liz-Marzán, SERS and plasmonic heating efficiency from anisotropic core/satellite superstructures, Nanoscale, 11 (2019) 17655-17663.
L. He, M. Brasino, C. Mao, S. Cho, W. Park, A. P. Goodwin, J. N. Cha, DNA-Assembled Core-Satellite Upconverting-Metal-Organic Framework Nanoparticle Superstructures for Efficient Photodynamic Therapy, Small, 13 (2017).
Q. Xiao, X. Zheng, W. Bu, W. Ge, S. Zhang, F. Chen, H. Xing, Q. Ren, W. Fan, K. Zhao, Y. Hua, J. Shi, A Core/Satellite Multifunctional Nanotheranostic for in Vivo Imaging and Tumor Eradication by Radiation/Photothermal Synergistic Therapy, Journal of the American Chemical Society, 135 (2013).
R. P. M. Höller, M. Dulle, S. Thoma, M. Mayer, A. M. Steiner, S. Förster, A. Fery, C. Kuttner, M. Chanana, Protein-Assisted Assembly of Modular 3D Plasmonic Raspberry-like Core/Satellite Nanoclusters: Correlation of Structure and Optical Properties, ACS Nano, 10 (2016) 5740-5750.
C. Wong, T. Stylianopoulos, J. Cui, J. Martin, V. P. Chauhan, W. Jiang, Z. Popovic, R. K. Jain, M. G. Bawendi, D. Fukumura, Multistage nanoparticle delivery system for deep penetration into tumor tissue, Proceedings of the National Academy of Sciences, 108 (2011).
X. Cheng, H. Li, X. Ge, L. Chen, Y. Liu, W. Mao, B. Zhao, W.-E. Yuan, Tumor-Microenvironment-Responsive Size-Shrinkable Drug-Delivery Nanosystems for Deepened Penetration Into Tumors, Frontiers in Molecular Biosciences, 7 (2020).

### SUMMARY OF THE INVENTION

A preferred embodiment provides method for synthesizing ultrasmall gelatin nanoparticles. A first desolvation is conducted to produce gelatin strands in a first solution and then setting the pH of the solution to charge and separate the strands. Tripolyphosphate (TPP) is added to the first solution to form gelatin-TPP-gelatin bridges. Second desolvation of the gelatin-TPP-gelatin bridges is conducted in a second solution. The second solution contains an TPP-Ethanol:Acetone mixture in a ratio between 1:1 and 1:5 and TPP between 0.005-0.025 vol % or a Glutaraldehyde-Ethanol:Acetone mixture in a ratio of glutaraldehyde-ethanol:Acetone range of 2.5-12% v/v to produce a colloid of self-assembled gelatin nanoparticles.

Preferred methods can produce nanomaterial consisting of a plurality of gelatin nanoparticles sized at ˜10 nm. The nanomaterial can encapsulate a drug, metal nanoparticle or contrast agent.

### BRIEF DESCRIPTION OF THE DRAWINGS

FIG. 1 illustrates preferred methods of the invention for synthesizing ultrasmall gelatin nanoparticles;

FIGS. 2A-2E show: HR-TEM images of solution during early-stage of synthesis of gelatin nanoparticles with formation of primordial toroid-like nanoparticles indicated with arrows (FIGS. 2A-2B); HR-TEM images of purified gelatin nanoparticles showing uniform sized-nanoparticles (FIG. 2C); DLS spectra (FIG. 2D) Zeta potential spectra (FIG. 2E) for gelatin nanoparticles showing ˜10 nm hydrodynamic size with positive zeta potential; and

FIGS. 3A and 3B show data concerning (A) optimization of TPP concentration in nanoprecipitant-mixture added to reaction solution for formation of G^{X} and (B) optimization of Ethanol:Acetone ratio (nanoprecipitant-solution) added to reaction solution for formation of G^{X}.

### DETAILED DESCRIPTION OF THE PREFERRED EMBODIMENTS

Preferred methods of the invention modify the traditional two-step desolvation. Prior to a second desolvation, some cross-linking with tripolyphosphate (TPP) is conducted to form gelatin-TPP-gelatin bridges. Second desolvation and cross-linking is then conducted in a second solution that contains an TPP-Ethanol:Acetone mixture in a ratio between 1:1 and 1:5 and TPP between 0.005-0.025 vol % or a Glutaraldehyde-Ethanol:Acetone mixture in a ratio of glutaraldehyde-ethanol:Acetone range of 2.5-12% v/v to produce a colloid of self-assembled gelatin nanoparticles. Further steps can isolate different sizes of gelatin nanoparticles, including nanoparticles consisting of 10 nm. Drugs or other payloads can be added prior ot the second desolvation.

Current processes and particles are based upon analysis and unique insight of the present inventors about prior processes. In conventional two-step desolvation processes to produce large, e.g., 200 nm particles, the first step involves separation of low and high molecular weight gelatin fractions, and the subsequent step is the acidification of the high molecular weight fraction solution to protonate the amines in the gelatin backbone to form ammonium ions. These cationic fragments induce repulsion between gelatin units to keep them apart. Addition of antisolvent to this solution, precipitates to form gelatin nanoparticles of ˜200 nm. The acidification step controls the phase behavior of gelatin sol and allows homogenous nanoprecipitation by displacing water molecules between units. The individual units are clustered together with high degree of randomness; therefore, the nanoprecipitation step results in larger size gelatin clusters. We hypothesized that if the positively charged gelatin strands are arranged in a systematic fashion then the desolvation step would induce smaller non-random clusters. We determined that directionally connecting the ammonium ions within the same strand using an anionic crosslinker to form a small ring-shaped (toroid-like) molecule would make the molecules coordinatively unsaturated, so they will act as nucleus to self-assemble with other units to form a smaller nanoparticle. With this approach, we conducted experiments to confirm this mechanism. We stabilized the intramolecular interaction of gelatin units using a polyanion that bridges an ionic link between the charged amine groups of two individual polymer units and slowed the nanoprecipitation kinetics using a mixture of antisolvents with different desolvation efficiencies. Specifically, anti-solvents such as ethanol and acetone are used to remove water molecules between gelatin molecules in a solution causing them to come close together and form nanoparticles.

Traditionally, desolvation is used to produce nanoprecipitates that are later crosslinked to form larger size nanoparticles. We have created a method for controlled formation of smaller nanoparticles via crosslinking gelatin during the second-desolvation step (while nanoprecipitation takes place) and not after. TPP is used to form controlled and shorter bridged structures. Other covalent crosslinkers would be non-specific and uncontrolled; and form permanent “amide” bonds between gelatin structures leading to larger particle sizes.

A preferred method for synthesizing ultrasmall gelatin nanoparticles includes cross-linking gelatin from a homogenous and acidified gel sol with sodium tripolyphosphate to form a gel solution. The gelatin nanoparticles are precipitated from the gel solution via addition of more than 99% volume ethanol:acetone with less than 1% volume of sodium tripolyphosphate to form a colloid. The colloid is stirred, cooled and then filtered. It is reheated then rapidly cooled in water. Rapid cooling can be conducted with cooling solution between 10-20 C. The solution is rapidly mixed (within 5-10 seconds) and kept at room temperature for 72 hours. This settles into a gel nanoparticle solution with a top solution having the ultrasmall nanoparticles and a bottom solution being a dense gel. The top solution via centrifugation forms fractional solutions. Dialyzing a selected one of the fractional solutions obtains an ultrasmall gelatin nanoparticle solution with gelatin nanoparticles sized less than 50 nm. In experiments, the components for dialysis included a 10 kDa dialysis tube containing the nanoparticle fraction kept in a beaker containing water for buffer exchange, and a 5% fraction was dialyzed. Preferred methods demonstrated solutions consisting of gelatin nanoparticles of 10 nm.

Preferred methods of the invention use a tripolyphosphate (TPP) crosslinker prior to the second desolvation, which is short enough to permit ammonium ions within the same strand to attach to one another rather than to different strands. Preferred methods use TPP to first form a gel solution, which permits the ammonium ions within the same strand to attach to one another rather than to different strands. Methods of the invention rely upon formation of Gelatin-TPP-gelatin bridge prior to second desolvation and controlling cluster size during cross-linking in the second desolvation using an Ethanol:Acetone mixture in a ratio between 1:1 and 1:5 and containing TPP (between 0.005-0.025% and preferably ˜0.01% volume), with a preferred ratio being between 1:1 and 1:2, and the most preferred ratio being 1:1. To synthesize stable and homogenous 10 nm NP, a range between 1:1 to 1:2 was found to be optimum. The ratio of acetone can be increased with some effect on stability.

Additional features and aspects of the invention will be apparent to artisans based upon the experiments that were conducted, which are presented next.

Materials. Gelatin (Bloom type-A), Sodium tripolyphosphate (TPP), Rhodamine-B, Cisplatin hydrochloride, sodium borohydride and glutaraldehyde were purchased from Sigma-Aldrich (USA). Sterile ultraclean distilled water, sodium hydroxide (NaOH), hydrochloric acid (HCl), 200-proof ethanol, acetone, sodium chloride (NaCl), sucrose, paraformaldehyde (PFA), RPMI-media, fetal bovine serum (FBS) and sodium borohydride were purchased from Thermo Fisher Scientific (USA). SH-Peg-OMe was purchased from RAPP Polymere (Germany) and SH-Peg-COOH was purchased from Laysan Bio (USA). Doxorubicin was purchased from LC Laboratories (USA). Cy-5 NHS ester was obtained from Lumiprobe (USA). Iodixanol (Visipaque®) was obtained from the University of Missouri Hospital.

### Synthesis of Ultra Small Particles G^{X}.

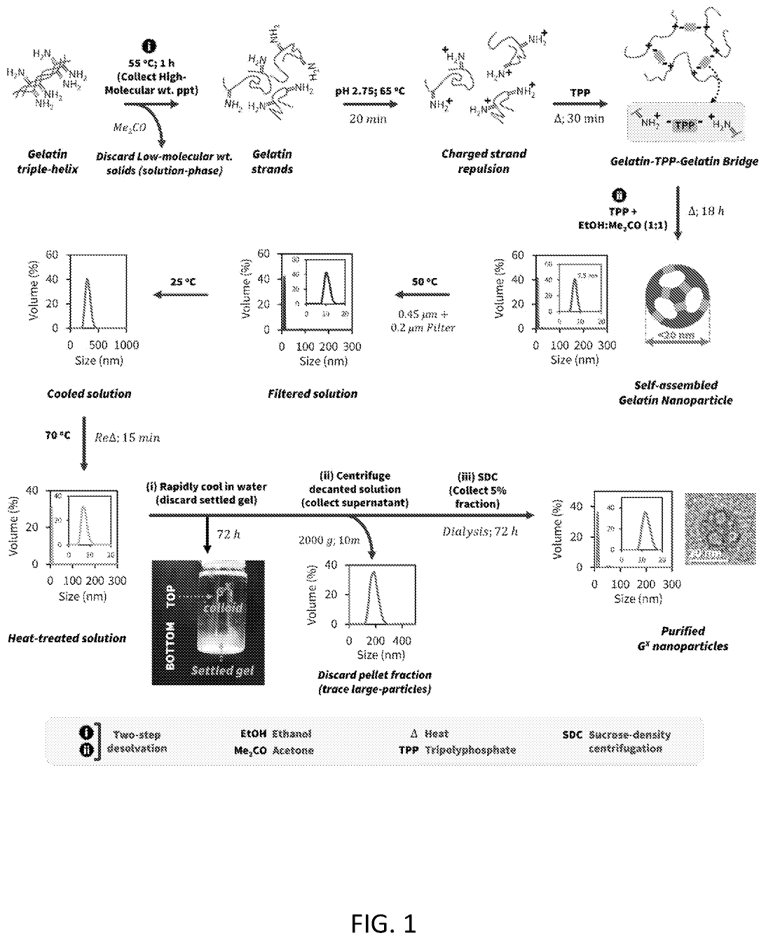

Linked Gelatin Strand Formation/First Desolvation. With reference to FIG. 1, gelatin strands are formed via first desolvation and then separated by setting the pH level to less than 2.8, e.g. 2.75. An acceptable range for pH is between 2.69-2.79. Then cross-linking is conducted in a second desolvation. The process to form the strands in an example experiment was as follows. Distilled water (3 ml) was heated in a 10 ml beaker (55° C.; 800 RPM) (permissible 55-65° C.). Gelatin powder (125 mg) was slow added to the heated water to avoid clumps and stirred until a homogenous gelatin sol was formed (˜ 1 h). For proper dissolution of gelatin in solution, a minimum time of one hour is required. During experimentation, solutions were kept up to 2 hours without any noticeable issues. More than 3 hours may allow evaporation to occur, leading to change in gelatin concentration. Next, acetone (6.5 ml) was rapidly added to the gel sol (900 RPM) and stirred for 30 s to precipitate high molecular weight (M.W.) solid fraction of gelatin. The supernatant containing low M.W. fraction was removed using a pipette and the precipitate was washed once with water (5 ml). Water (3 ml) was added to the beaker followed by gently loosening the precipitate using a pipette tip. The precipitate was then heated to form a gel sol again (55° C.; 800 RPM; 2 h)) (permissible 55-65° C.). As mentioned above, 1-3 h is an acceptable time period. After 2 h, the homogenous gel sol was acidified to pH 2.75 (1M HCl) and the solution was transferred to a 25 ml round-bottom flask (RBF) and allowed to stabilize at 65° C. (65-75° C. is acceptable) in an oil bath (900 RPM; 20 min). The second desolvation can then be conducted with a TPP crosslink or a Glutaraldehyde cross link.

TPP Crosslink. Tripolyphosphate (TPP; 0.5% w/v in water; 10 μl) was vortexed mixed with ethanol (190 μl). The TPP solution (total volume of 200 μl) was then added dropwise to the RBF at 10 mlh⁻¹ using an automated pipette fixed with a 10 μl pipette tip (for a slow controlled addition, the size of drop was kept small. Rates between 10-20 mlh⁻¹ are permissible) and the solution was stirred for 30 min. Next, tripolyphosphate (TPP; 0.5% w/v in water; 200 μl) was vortexed mixed with an ethanol:acetone mixture (1:1 v/v; 0.01% TPP) and added dropwise to the RBF at 25 mlh⁻¹ using an automated syringe pump (10 ml syringe fitted with a 15 mm acetone-resistant tube tipped with a 22 g needle). The solution color changed from transparent to a whitish colloid (˜10 ml of nanoprecipitant) at which precipitant addition was stopped and the solution was stirred for 18 h. After 18 h, solution was cooled (25° C.; 1 h) (permissible 25-35° C.; 1-2 h) and filtered through a 0.45 μm and a 0.22 μm sterile filter. The filtered solution was then reheated (70° C.; 15 m) (permissible 65-75° C.; 15-30 min), followed by rapid cooling (25° C.) by quickly mixing the reaction solution into a beaker containing water (2-3× volume; 800 RPM (water temp 10-20° C.)). The solution was allowed to cool and sit stationary for 72 h (permissible up to 96 hours), forming a dense irreversible gel that settled at the bottom of the solution. The top solution containing colloidal nanoparticles was decanted and purified using sucrose density centrifugation (SDC; Order: bottom—50%, 20%, 10%, 5%, 2%, top—reaction solution). The 5% fraction was then isolated and dialyzed (10 kDa) (permissible 5-50 kDa) in water for 72 h (permissible up to 96 h). Final solution containing nanoparticles was stored at 25° C.

Glutaraldehyde Crosslink. Ethanol:acetone mixture (1:10) was added to the RBF (25 mlh⁻¹) containing acidified gel sol until a white opaque colloid formed. Next, the solution was heated to 150° C. (permissible 140-150° C.) until a translucent whitish solution formed. Glutaraldehyde (25 μl; 25% v/v) mixed with ethanol (175 μl) was added dropwise and the reaction was stirred (900 RPM; 55° C.) (permissible 55-65° C.) until 18 h (permissible up to 24 h). This solution also formed ˜10 nm nanoparticles.

### Larger Gel Nanoparticles G^{L}.

Larger 50 nm gel nanoparticles G^{L} were formed. distilled water (5 ml) was heated in a 25 ml beaker (50° C.; 800 RPM) (permissible 50-60° C.). Gelatin powder (mg) was slow added to the heated water to avoid clumps and stirred until a homogenous gelatin sol was formed (˜ 1-2 h). Next, acetone (12 ml) was rapidly added to the gel sol (900 RPM) and stirred for 30 s to precipitate high molecular weight (M.W.) solid fraction of gelatin. The supernatant containing low M.W. fraction was removed using a pipette and the precipitate was washed once with water (5 ml). Water (5 ml) was added to the beaker followed by gently loosening the precipitate using a pipette tip. The precipitate was then heated to form a gel sol again (50° C.; 800 RPM; 2 h) (permissible 50-60° C. and up to 3 h). After 2 h, the homogenous gel sol was acidified to pH 2.75 (1M HCl) and the solution was transferred to a 50 ml round-bottom flask (RBF) and allowed to stabilize at 55° C. (permissible 55-65° C.) in an oil bath (900 RPM; 20-40 min). An ethanol:acetone mixture (1:10) was then added dropwise to the RBF at 50 mlh⁻¹ using an automated syringe pump (10 ml syringe fitted with a 15 mm acetone-resistant tube tipped with a 21 g needle) until a whitish colloid formed. Separately, tripolyphosphate (TPP; 0.5% w/v in water; 1 ml) was vortexed mixed with ethanol (1 ml). The TPP solution was then added dropwise to the RBF at 10 mlh⁻¹ using an automated pipette fixed with a 10 μl pipette tip until a slight milkish color formed. Glutaraldehyde (10 μl; 25% v/v) mixed with ethanol (190 μl) was added dropwise and the reaction was stirred (800 RPM; 55° C.) (permissible 55-65° C.) until 18-20 h. After 18 h, solution was cooled (25° C.; 1 h) and filtered through a 0.45 μm sterile filter and centrifuged to collect the pellet (15,000 g, 15 min). Pellet was resuspended in water using sonication and washed two times (15,000 g, 15-20 min as long as a pellet is identified visually at the bottom of the tube along with a clear-supernatant) and the final resuspension in water containing nanoparticles was stored at 20-25° C.).

Very Large Gel Particle Formation G^{CC}. Very large 200 nm gel nanoparticles were formed using a conventional two-step desolvation procedure. Distilled water (11 ml) was heated in a 100 ml beaker (50° C.; 800 RPM). Gelatin powder (500 mg) was slow added to the heated water to avoid clumps and stirred until a homogenous gelatin sol was formed (˜ 1 h). Next, acetone (20 ml) was rapidly added to the gel sol (900 RPM) and stirred for 30 s to precipitate high molecular weight (M.W.) solid fraction of gelatin. The solution containing low M.W. fraction was removed using a pipette and the precipitate was washed once with water (5 ml). Water (11 ml) was added to the beaker followed by gently loosening the precipitate using a pipette tip. The precipitate was then allowed to form a gel sol again (50° C.; 800 RPM; 2 h). After 2 h, the homogenous gel sol was acidified to pH 2.75 (1M HCl) and the solution was transferred to a 100 ml round-bottom flask (RBF) and allowed to stabilize at 50° C. in an oil bath (800 RPM; 10 min). 100% Acetone was then added dropwise to the RBF at 100 mlh⁻¹ using an automated syringe pump (10 ml syringe fitted with a 15 mm acetone-resistant tube) until a whitish colloid formed (800 RPM; 10 m). Next, glutaraldehyde (200 μl; 25% v/v) was added dropwise and the reaction was stirred (800 RPM; 50° C.) until 18 h. After 18 h, solution was cooled (25° C.; 1 h) and centrifuged to collect the pellet. Pellet was resuspended in water using sonication and washed four times (20,000 g, 20 min) and the final resuspension in water was filtered through a 0.45 μm sterile filter. Final solution containing nanoparticles was stored at 4° C.

Dye tagging on GNP. To tag Rhodamine-B (RhB) or Cy-5 NHS ester, 2 mg of dye solution in water (1 ml) was added dropwise to G^{X} or G^{L} or G^{CC} solutions and allowed to stir (800 RPM; 18 h; 25° C.). For Cy-5 tagging the reaction solution pH was adjusted to 8.2 (1 M NaOH). After 18 h, G^{X} was subjected to dialysis in water for (96 h) while G^{L} and G^{CC} were purified using centrifugation. Final construct solutions for GNP-RhB or GNP-Cy5 were stored at 25° C.

Encapsulation of Drugs or Contrast agent. To encapsulate drugs such as doxorubicin (DOX), and cisplatin (CP) or contrast agents such as iodixanol (IO), 5 mg DOX in water or 5 mg CP in 0.9% NaCl solution, or 10 mg iodixanol solution was dropwise added to the gel sol before the acidification step. After synthesis and purification of the G^{X} solution, final solutions for G^{X}(DOX) or G^{X}(CP) or G^{X}(IO) were stored at 25° C.

Synthesis of G^{X}(Au) Hybrid Nanocomposite. For synthesizing multi-dimensional hybrid materials 2 nm AuNP coated with either thiol-ligand or NaBH₄ or SH-PEG-COOH (M.W. 2000) or THPC; 10 nm AuNP coated with citrate were used. Gold nanoparticles were observed on the surface of Gx. To encapsulate gold nanoparticles of various sizes, 1 mg of AuNP in water was mixed with the gel sol before the acidification step for 1-2 h. After synthesis and purification of the G^{X} solution, final solutions for G^{X}(AuNP) were stored at 25° C. Other metal nanoparticles, e.g., silver nanoparticles can be used in instead of gold. Specifically, any metal nanoparticle less than ˜3 nm in diameter, with a hydrophilic coating (short ligand; not long chained) can be encapsulated within Gx. For example, silver nanoparticles with such properties can be encapsulated.

Synthesis of G^{CC}_G^{X}-satellite Hybrid Nanocomposite. To synthesize G^{CC} particles with G^{X} satellites, G^{X} nanoparticles were first synthesized and passed through a 0.2 μm filter. Next, this solution was reheated (65° C.) and a mixture containing TPP (50 μl; 0.5% w/v), Glutaraldehyde (5-10 μl; 25% w/v) and SH-Peg-Ome (0.1 mg; M.W. 750) diluted in 1 ml of Ethanol was added. Reaction was stirred for 72 h forming an off-white color solution indicating formation of G^{CC}-G^{X}sat hybrid particles. Final solutions for were stored at 25° C.

Dynamic Light Scattering (DLS). To monitor particle size, dynamic light scattering (DLS) technique using a Non-invasive backscatter technology was used. Measurements were performed on a Zetasizer rated for 0.3 nm-10 μm, using a 633 nm He—Ne laser source with a backscattering angle (NIBS) of 173°. Briefly, a 0.05 mg/ml GNP solution in water was prepared and 800 μl of this solution was sonicated (10 s), vortexed (5 s) and added into a low-volume cuvette. For the sample parameters, material was selected as protein with a refractive index of n=1.45, solvent was water and once the temperature was equilibrated to 25° C., high-resolution measurements were performed in triplicates (each measurement performed using 3 runs, 10 s each). The data analysis was performed on the software suite and the particle size data values were exported to excel for plotting.

Zeta Potential Measurement. To monitor particle surface charge, laser Doppler microelectrophoresis technique using a Non-invasive backscatter technology was used. Measurements of the surface charge at slipping planes (zeta potential) were determined on a Zetasizer using a 633 nm He—Ne laser source for conducting electrophoresis light scattering (ELS). Briefly, a 0.05 mg/ml GNP solution in water was prepared and 900 μl of this solution was sonicated (10 s), vortexed (5 s) and added into a DTS-1070 Zeta-measurement cell. For the sample parameters, material was selected as protein, Debye-Huckel approximation, solvent was water and once the temperature was equilibrated to 25° C., measurements were performed in triplicates (each measurement performed using 10 runs). The data analysis was performed on the software suite and the particle size data values were exported to excel for plotting.

High-Resolution Transmission Electron Microscopy (HR-TEM). To image GNP, high-resolution transmission electron microscopy (HR-TEM) was used. For this purpose, GNP stock solution (40 μl) was rapidly mixed in water (200 μl) using vortex mixing. The solution was then sonicated (5 s) and 8 μl was dropped on a 200-mesh carbon-coated copper grid. The drop was then air-dried (40° C.; 10 m). The air-dried grids were then inserted into the HR-TEM using a single-sample single-tilt holder and imaged at 100 kV. Beam alignment (pivot point X and Y; shift and rotation center), coma-free beam alignment, and sigmator-alignment (condenser and objective) were performed at both low (<10 μm×10 μm) and high (<100 nm×100 nm) magnification prior to image acquisition. At 200 kV particle disintegration was observed for long beam exposures (>1 m). Images were taken using a 1-0.5 s exposure and converted to TIFF using Gatan software.

Energy Dispersive Spectroscopy (EDS). To detect elements in G^{X}(CP) or G^{X}(IO), Scanning transmission electron microscopy—Energy-dispersive X-ray spectroscopy (STEM-EDS) was used. To perform measurements, carbon-coated copper grids with air-dried nanoparticles were inserted using a single-sample low-background double-tilt holder. After, STEM alignment, STEM microscopy was performed at 200 kV in high angle annular dark field (HAADF) imaging mode. The STEM-EDS map acquisition were then performed using a 30 mm² active area Bruker Silicon Drift Detector with a super light element window. Final spectral plots were then collected and saved using Bruker ESPIRIT software.

Scanning Transmission Electron Microscopy (STEM). To image GNP, scanning transmission electron microscopy (STEM) was used. Briefly carbon-coated copper grids with air-dried nanoparticles were inserted using a multi-sample stage and imaged between 5-20 kV using the STEM detector.

Image Analysis and Volume Reconstruction. To predict the three-dimensional (3D) structure of G^{X} nanoparticles we partially reconstructed the 3D topography inferred from the TEM images, and prepared iterative representative 3D volume models. In brief, the TEM images were converted to 32-bit and processed for background subtraction, contrast enhancement, distance map to distance ridge mapping, and Euclidean distance transformation. This allowed the de-noising and selective computation of pixel intensities to visualize distance. Further the maps were processed using ImageJ Process-plugin to calculate the maximum 3D points to refine local thickness. Subsequently, the maximum values were used to build 3D surface plots to analyze surface topography of the TEM images. Using a 3-D builder the topography was approximated to a block and duplicated along the z-axis to visualize a 3D structure. Using visual cues of the vertexes and pore locations, several iterative 3D models were prepared using 3-D builder. Each of these models were rotated and several projected planes were captured emulating a two-dimensional (2D) image. The emulated 2D images were processed through similar distance mapping and representative 3D surface plots were created. The predicted 3D surface plots were matched with the observations to select approximate structures that represent G^{X}. Final maps were saved as TIFF using ImageJ (NIH).

3D Spheroid Assay. To perform the spheroid penetration assay, NCI-ADR-RES or A549 cell lines (90% confluency) were used. Briefly, three hundred A549 or NCI-ADR-RES cells were suspended in 200 μl of complete medium (RPMI 1640+10% FBS) and seeded into a Corning 96-well ultralow attachment microplate (Corning 4520), followed by a centrifugation (200 g; 30 s; 25° C.). The plate was then incubated at 37° C.; 5% CO2 for 5 days to allow the formation of spheroids (triplicates). The spheroids were washed twice with serum free media and treated with nanoconstructs (0.4 mg/ml; 14 h; 37° C.) to evaluate tumor penetrability. The treated spheroids were washed thrice with 1×DPBS and fixed in 2% buffered PFA (2 h). Fixed spheroids were washed thrice with DPBS and transferred onto confocal dishes for acquisition of confocal z-stacks of 3D spheroids using confocal fluorescence microscopy.

Confocal Fluorescence Microscopy (CFM). To analyze fluorescence in spheroids, confocal dishes containing untreated and treated spheroids were imaged using a Leica TCS SP8 Confocal microscope (Leica Application Suite X software) using the excitation/emission parameters of rhodamine-B (554/564-644 nm) and Cy-5 signal (647/655-705 nm). Frame Average 1, Line Average 3, Frame Accumulation 1, Line Accumulation 1, Linear Z Compensation was applied. Z-sections at spheroid depths of 10, 20, 30, 40, 50 and 60 μm were analyzed to study penetration between constructs. Spheroid segmentation was carried out using the SpheroidJ plugin. [2] The inner region of each section was defined by ⅔^{rd} of the radius. Mean intensity calculation of the inner and outer region of each section was carried out using Fiji (ImageJ). [3]3D reconstruction of spheroids was performed using z-scan stacking and rotation along the x-axis using Leica Application Suite X software.

Statistical Analysis. All data was produced from triplicates and averaged unless indicated. Data was expressed as Mean±Standard error of the mean. Statistical significance was computed using student t-test, one-way or two-way analysis of variance (ANOVA) using Tukey method on Prism software (GraphPad, San Diego, CA, USA). P-values <0.05 at 95% confidence interval were considered significant.

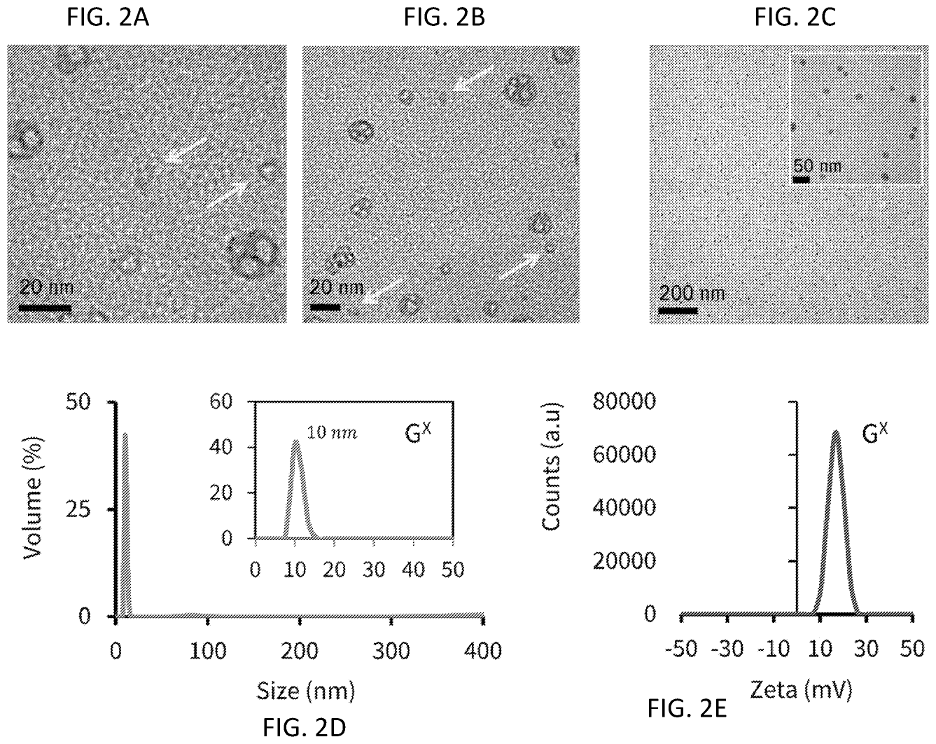

Formation Mechanism Confirmation. To verify our formation mechanism hypothesis discussed above, we treated the acidified solution of gelatin with sodium tripolyphosphate (Na₅P₃O₁₀; TPP), a sodium salt of penta-anion polyphosphate to spatially entrap gelatin strands to form nanoclusters. Bridge describes interaction between two gelatin strands mediated by TPP, while nanoclusters are larger than two gelatin strands to form a larger cluster of gelatin molecules bound by TPP. Upon treatment of the nanoclusters with Ethanol-Acetone mixture (v/v; 1:1) ˜5 nm “nucleus” nanoparticles are formed within few minutes of the reaction as evidenced by HR-TEM images as shown in FIGS. 2A and 2B. These nanoparticles resemble toroid-like ring structures. After the completion of the reaction, the toroid structures assembled to form stable 10 nm sized larger polyhedral structures. We believe the intermediate toroid-like units may play a role in the hierarchical self-assembly process to create the higher-order geometries. To confirm that phosphates in TPP mediated the assembly process, we performed STEM-EDS spectra of the 10 nm size gelatin nanoparticle, and it showed the presence of phosphorus within the framework. Further, by controlling the gel-sol:Nanoprecipitant (3:10 v/v) ratio, a homogenous solution composed of positively charged ˜10 nm porous hollow structures (G^{X}) were synthesized as indicated in FIGS. 2C-2E. The nanoparticle structure resembled a complex class II geodesic polyhedron with rotational vertexes, thickness around 1.5 nm with a large cavity around 5-10 nm. The hydrodynamic mean-diameter of purified G^{X} is 10±3 nm with a zeta potential of ±20 mV.

3D Shape of G^{X}. We performed an in-depth image analysis on understanding the 3D geometry of G^{X} to obtain insight into the self-assembly of these nanoparticles. In the first step, we used the particles' HR-TEM images to map the local thickness and distance between pixels within the image. Further using the resolved distance-maps, we created 3D surface plots at various angles. We then prepared iterative representative volumetric models and converted the models to 3D surface plots to select approximate models that best represent G^{X} morphology. The analysis showed that the structures were polyhedron in nature with rotational vertexes. Based on the reconstructed volumetric models, we predict that each higher order G^{X} nanostructure is self-assembly of 4 or 5 toroid-like units. The self-assembly is initiated by TPP molecules by bridging with ammonium ions in gelatin strands and facilitated by controlled removal of water molecules adjacent to these units in the presence of antisolvent. The self-assembly process creates a porous void in the 3D structure. The size and density of these structures indicated that the G^{X} particles could quickly scatter incident photons, thus could be monitored using the dynamic light scattering (DLS) technique.

Optimization of G^{X} Synthesis. We optimized all parameters, using DLS, including TPP concentration, antisolvent ratios, and pH to obtain homogeneous G^{X} particles. We considered with regards to the use of TPP: (i) Whether the TPP should be added before or after the desolvation step? (ii) and, what is the optimum amount of TPP required to obtain uniform size G^{X}? To answer to the first question, we performed a series of reactions wherein we added TPP before or after the nanoprecipitation (or desolvation) step. If the antisolvent precipitates gelatin, then the molecular arrangement will be disrupted and TPP would become incapable to form ionic bridges. As predicted, the addition of TPP after desolvation led to formation of non-homogenous nanoparticle populations with larger sizes. In sharp contrast, as predicted, addition of TPP before the precipitation step results in uniform size G^{X} formation. The study further confirms that TPP should be added before desolvation as it mediates the ionic bond to enable the self-assembly process and generate uniform size G^{X}. To answer our second question, we systematically increased the concentration of TPP (0; 0.1; 0.4; 1 or 2.5 mg/ml) in the nanoprecipitant solution and added it at the second desolvation step. The results show that the up to a certain concentration (0.4 mg/ml) the reaction is resilient, and the size of the particle showed no variation.

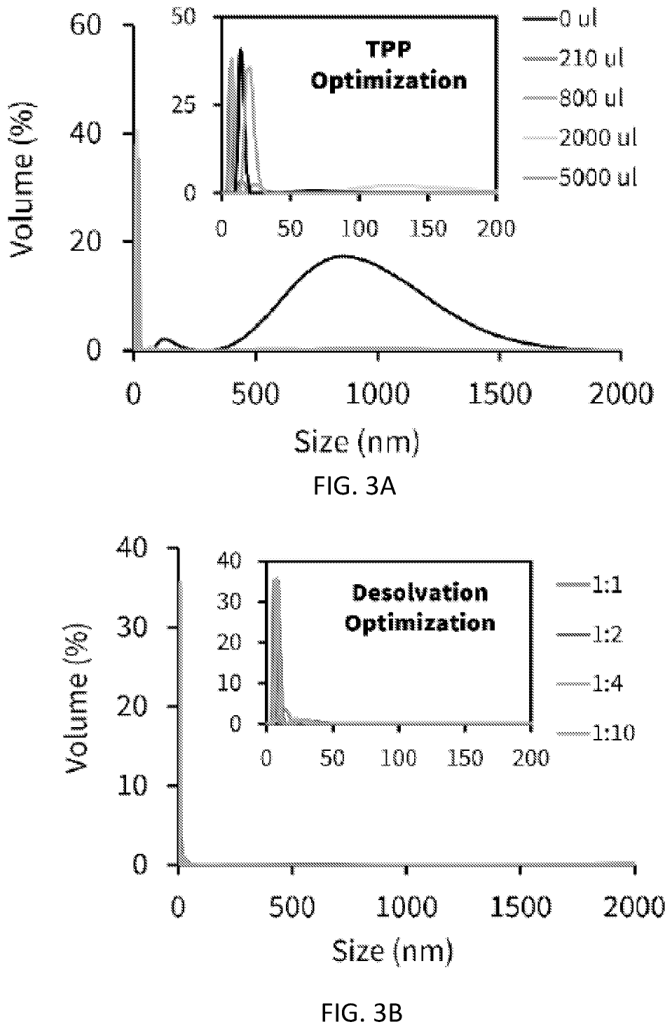

After the threshold concentration the gelatin particles become destabilized and precipitates from the reaction, possibly due to salting-out principle. Based on our experiments, we found that ˜200 μl of 0.5% TPP in the nanoprecipitant solution (0.1 mg/ml) is optimum to form a uniform size G^{X}, as seen in FIG. 3A and the following table.

| | | Hyd. | | | Nanopre- | | |
|---|---|---|---|---|---|---|---|
| TPP | | Size | | | cipitant | | |
| (0.5%) | | (nm) | SE | Pdl | (ml) | Observation | Stability |
| 0 | μl | 644.3 | 104.8 | 0.5 | 13.0 | Whitish Opaque | Small ppt |
| 210 | μl | 7.9 | 0.8 | 0.4 | 10.0 | Whitish Opaque | Colloidal |
| 800 | μl | 9.7 | 1.0 | 0.8 | 6.0 | Whitish | Turbid |
| | | | | | | Translucent | |
| 2000 | μl | 14.0 | 0.6 | 0.8 | 5.0 | Glassy | Small ppt |
| | | | | | | Translucent* | |
| 5000 | μl | 20.3 | 3.2 | 0.5 | 4.0 | Glassy* | Large ppt |

*Higher TPP reactions did not require reheating

As a next step, we studied the influence of desolvation mixture, ethanol:acetone, in the size of the G^{X} nanoparticles. We used and the following ratios—1:1; 1:2; 1:4 or 1:10 (EtOH:Me₂CO) to precipitate the nanoparticles and monitored the size by DLS. Both 1:1 and 1:2 showed no variation in the final G″ formation, and the sizes are uniform. However, increasing the ratio of acetone to 1:4 or 1:10, results in the turbidity indicating the aggregation of gelatin. These results agreed with the previous literature which has shown denaturation led to increased particle size. Based on the results, it is apparent that a 1:1 ethanol:acetone mixture was optimum in producing a homogenous colloidal solution without any visible precipitation as shown in FIG. 4B and the following table.

| | Hyd. | | | Nano- | | |
|---|---|---|---|---|---|---|
| | Size | | | precipitant | | |
| Ethanol:Acetone | (nm) | SE | Pdl | (ml) | Observation | Stability |
| 1:1 | 7.9 | 0.8 | 0.5 | 10.0 | Whitish | Colloidal |
| | | | | | Opaque | |
| 1:2 | 7.5 | 0.4 | 0.7 | 9.0 | Whitish | Colloidal |
| | | | | | Opaque | |
| 1:4 | 8.2 | 1.4 | 0.9 | 6.0 | Whitish | Turbid |
| | | | | | Opaque | |
| 1:10 | 8.0 | 1.0 | 0.9 | 5.0 | Whitish | Turbid |
| | | | | | Opaque | |

pH Adjustment. The pH of the reaction determines the number of protonated amines in the backbone of gelatin. The pI of the gelatin type-A that we used in the study is between pH 7-9. Therefore, we chose to study the influence of pH (2, 2.75, and 5.6) in the size and homogeneity of generated GN nanoparticles. If the pH of the solution is <6 (acidic) then the particles are smaller in size. Even though the smaller particles are formed at pH 5.6, the solution was glassy, and the precipitation ensued rapidly. The precipitation may be due to lower repulsion between gelatin units. On the other hand, lowering the pH≤2 required more antisolvent to form nanoparticles, thereby changing the nanoprecipitant:gelatin ratios and causing visible destabilization. Based on our data, we found that pH 2.75 is optimum and pH range 2.69-2.79 is suitable to form G^{X}. Finally, we studied the effect of salt (10 mM NaCl) or sugar (10% sucrose) in gel sol in the formation of nanoparticles. The results showed that formation of G^{X} in unhindered by either of the molecules further reiterating the H-bonding is between ammonium ions and TPP molecules.

Excellent in vitro Spheroid Penetration by G^{X}. We examined the effects of the size of GNP on the penetration into tumors. GNP contains rich functional groups to conjugate or attach dye molecules; in this study, we surface adsorbed Rhodamine-B (RhB) or covalently conjugated Cyanine-5 (Cy5) to form G^{M}-dye (M=X (10 nm), L (50 nm), or CC (200 nm); dye=RhB or Cy5) constructs (see ES). The conjugates were well characterized using TEM, DLS, zeta, and fluorescence measurements. We developed both ovarian and non-small cell lung cancer (NSCLC) spheroids based on the manufacturer's protocols. The spheroid formation was monitored by bright-field microscopy. After 3 days of the culture, the spheroids were formed, and the sizes of 197±15 μm (NCI-ADR-RES) 173±12 μm (A549) were selected for the study. The size variation in spheroids grew in different plates were negligible. The cells were tightly packed in the spheroid formed from ovarian cancer cell (NCI-ADR RES) when compared with that of NSCLC (A549) cells.

The confocal fluorescence microscopy (CFM) can be used as a tool to evaluate the penetration of the fluorescent G^{M}-dye constructs in tumor spheroids. As a first step, we incubated the spheroids with different concentrations of free dye and imaged using CFM. As such, the dye fails to enter into cells, even with the very high concentrations of the dye a faint, scattered fluorescence was observed. We chose to use 10-fold lower concentrations of the dye containing nanoparticles for our experiment, thus, any fluorescence observed within the spheroid truly emanate from the nanoparticle and not from inadvertently released dye. Additionally, the chosen concentrations of the G^{M}-dye did not show any toxicity in cells and the spheroid integrity was maintained. After selecting the working concentration, the ovarian and NSCLC spheroids were incubated with G^{M}-dye for a period of 14 hours. The spheroids were imaged at different depths using CFM.

To provide a qualitative picture of the CFM data, we segmented the spheroid into three sections: outer (10-20 μm); inner (30-40 μm); and deep (50 μm). In case of NCI-ADR-RES, the G^{cc}-dye particles are brightly seen in outer spheroid, and faintly in inner spheroid, however, no fluorescence is observed in deep region. On the other hand, the G^{LL}-dye particles are prominent in inner region, faint in both outer and deep regions of the spheroid. In sharp contrast to the other two nanoparticles, G^{X}-dye particles traveled to deeper regions of the spheroid and the fluorescence is widely observed; however, the fluorescence is faint in both outer and inner regions of the spheroids. Based on this data, we can say that the nanoparticles show the following order of penetrance to deep tumors: G^{X}>>G^{L}>G^{CC}. As observed in mean normalized intensity at deep regions (50 μm depth), the G^{X} particles show higher fluorescence when compared with other two sizes.

Next, we performed a similar study in A549 spheroids, wherein, the cells are irregularly packed when compared with the other cells. We chose this type of packed cells as it provides an opportunity to evaluate whether the nanoparticles would behave the same. Interestingly, the trend that smaller particles reached deep regions of spheroid remained largely the same, but one anomaly was observed. In the case of rhodamine labeled dye particles, G^{L} showed higher fluorescence in deep sphere than G^{X}. But the G^{L}-Cy5 showed weak fluorescence in deep spheroid when compared with G^{X}. The Cy5 labeled nanoparticles follows the order for the transport in deep tumor: G^{X}>>G^{L}>G^{CC}. Results show that G^{X}-dye penetration was statistically significant (p<0.05) than G^{L}-dye or G^{CC}-dye constructs.

To obtain a comprehensive understanding, we performed spheroid segmentation wherein the inner region of each section was defined by ⅔^{rd} of the radius. Using the segmented data, we could analyze the relative mean intensity within the inner and outer regions of the section, thereby resolving the deep penetration achieved by the G^{M}-dye constructs. Result indicated that, on an average G^{X}-Dye penetrated higher than both G^{L} or G^{CC} constructs. We observed that the difference between the penetration depth of G^{X}-RhB and G^{L}-RhB was lower, while the difference was higher in G^{X}-Cy5 and G^{L}-Cy5. We attributed this to the dye attachment characteristics, as adsorbed RhB may be released while covalently conjugated Cy5 would be tethered to the GNP. In both NCI-ADR-RES and A549 spheroids, G^{CC}-dye constructs showed poor penetration and higher accumulation to the outer segment of the spheroid. We also stacked CFM images on z-axis, to view a 3D reconstruction within the spheroid (rotated along x-axis); the 3D volume shows higher G^{X}-Dye penetration within both NCI-ADR-RES or A549 tumor spheroids than G^{L}-RhB or G^{CC}-Dye constructs. It is important to note that, 200 nm G^{CC}-dye constructs formed a shell around outer layer of the tumor spheroids indicating high tumor-surface localization. Our results showed that smaller-sized particles penetrate deep into the tumor better than larger-sized particles

Entrapment of Chemotherapeutic Drugs within G^{x}. The TEM images and 3D volume reconstruction models show that G^{X} particles is a nanocapsule with a void inside. Further, the model showed that the nanocapsule shell has openings, probably populated with H-bonds. The structure of G^{X} intrigued as to understand whether it can encapsulate drug within the capsule. As the gelatin is ampholytic, the backbone can attach with both hydrophobic and hydrophilic molecules. We chose two drug molecules doxorubicin and cisplatin, for our studies to generate (G^{X}(R); R=Dox or CP). We mixed predetermined concentrations of the drug (mg) with protonated gelatin and followed the protocol for synthesizing G^{X}. After the synthesis, we centrifuged the reaction mixture and washed the pellet to remove the unreacted molecules. The hydrodynamic sizes of these particles were ˜ 12 to 20 nm with the PDI of ˜0.5 to 0.8. The cisplatin encapsulated G^{X} was slightly larger (˜19 nm). The zeta potential of G^{X}(R) was positive and greater than 20 mV. STEM-EDS spectra of G^{X}(CP) showed platinum peaks confirming that presence of drug trapped inside. We recorded the UV-Visible absorption/fluorescence spectra of G^{X}(DOX), and it showed a characteristic absorbance at 490 and emission at 590 nm. The study showed that the void in G^{X} is adequate to encapsulate drugs and still retain the smaller geometry.

Entrapment of gold nanoparticle within G^{x}. Owing to the unique size and morphology of G^{X}, we explored the potential of its use in synthesis of hybrid nanostructures. We studied the (i) formation of hybrid nanomaterials by entrapping gold nanoparticles (AuNP) within the cavity of G^{X}, and (ii) developing an in situ synthetic methodology for forming core-satellite hybrid nanoparticles exclusively using gelatin matrix.

For forming dual-nanoparticle G^{X}(Au) hybrid, AuNP was mixed with gel sol and then nanoprecipitated. As observed in HR-TEM images, 2 nm AuNP were effectively entrapped within the structure of G^{X} (˜10 AuNP/G^{X}). DLS spectra showed minimal change to the hydrodynamic size as the AuNP was inside rather than on the surface of G^{X}. The entrapment efficiency varied based on the surface coating on the AuNP. By changing the surface characteristics or size of AuNP, we observed AuNP would attach to the surface of G^{X} as seen in HR-TEM images and a correlated shift in DLS spectra. Such hybrid assemblies can be used to deliver ultra-small 2 nm gold nanoparticles deep within a tumor. Potentially, such assemblies can be functionalized, wherein each nanocomponent could carry a drug or contrast agent for efficient theranostics.

Hybrid Nanostructures. We developed a novel in situ synthesis where G^{X} and G^{CC} particles were synthesized in the same reaction solution with a mixture of crosslinkers. In this reaction, G^{X} was first synthesized followed by addition of a crosslinker mixture containing TPP and a covalent crosslinker glutaraldehyde (GLU). GLU is known to crosslink through the lysine e-amino groups and N-terminal amino acids of the gelatin chain and has been used in numerous studies for forming larger 200 nm G^{CC}. HR-TEM images showed the formation of G^{CC}-G^{X}sat core-satellite assemblies where 150-200 nm GNP were decorated with an average of 4 G^{X} particles. Prior attempts to form such core-satellite assemblies often involved metal-metal or metal-organic frameworks based on electrostatic or covalent linking methods. G^{CC} was formed in a G^{X} reaction solution and G^{X} was not separately attached. As such these multi-dimensional hybrids were self-assembled in situ and each nanocomponent (200 nm and 10 nm) may be used for efficient tumor localization and deep penetration. We also found that by changing the amount of glutaraldehyde in the reaction we could tune the size of the satellite nanoparticles, as seen in the following table.

| Sample | Hyd. Size (nm) | SE | Pdl |
|---|---|---|---|
| G^{X}(2 nm Au—NaBH₄) | 10.0 | 0.5 | 0.9 |
| G^{X}(2 nm Au-Peg) | 12.6 | 2.3 | 0.8 |
| G^{X}(2 nm Au-THPC) | 13.4 | 0.9 | 0.6 |
| G^{X}(5-10 nm Au-Citrate) | 17.5 | 4.3 | 0.8 |

| Sample | Zeta (mV) | SE | Pdl |
|---|---|---|---|
| G^{X}(2 nm Au—NaBH₄) | 27.00 | 1.47 | 0.9 |
| G^{X}(2 nm AuNP-Peg) | 27.13 | 0.68 | 0.8 |
| G^{X}(2 nm Au-THPC) | 26.20 | 0.24 | 0.6 |
| G^{X}(5-10 nm AU-Citrate) | 25.70 | 0.68 | 0.8 |

These results demonstrate the potential of dual-nanoparticle based hybrid nanostructures that can be used to increase the efficiency of tumor penetration and other applications where size-based staged delivery may be appropriate.

Ultra-Small Theranostic Delivery Device. To understand the relation between penetration efficiency and the size of the gelatin nanoparticles, we tagged the surface of the gelatin nanoparticles namely, G-10, G-50 and G-200 with rhodamine-B dye. By tagging the nanoparticles, it allowed us to monitor and observe the internalization of nanoparticles in an in-vitro setting. For this purpose, we grew spheroids from two different cancer cell lines namely NCI-ADR-RES (ovarian cancer cell line) and A549 (non-small cell lung cancer cell line). We treated these spheroids with rhodamine-tagged nanoparticles for 14 hours and imaged them using confocal microscopy. Fluorescence tagging allowed us to visualize the internalization of nanoparticles at the interface and within the spheroid at various depths. Using the data, we performed cross-section analysis at various depths and using z-stacking we reconstructed the spheroid data in 3D. Images showed the presence of rhodamine tagged G-10, G-50 and G-200 nanoparticles at various depths of the spheroids (from 10 μm to 50 μm). Results indicate higher accumulation of larger particles at the interface while showing higher penetration by smaller particles. A distinct shell was visually observable by G-200 at 50 μm indicating accumulation of particles at the surface of the spheroid.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A nanomaterial consisting of a plurality of gelatin nanoparticles sized at ˜10 nm.

2. The nanomaterial of claim 1, wherein the gelatin nanoparticles encapsulate gold nanoparticle cores sized at ˜2 nm.

3. The nanomaterial of claim 1, wherein the gelatin nanoparticles encapsulate iodine containing X-ray contrast agents.

4. The nanomaterial of claim 1, decorated on a surface of larger gelatin nanoparticles.

5. The nanomaterial of claim 4, wherein the larger gelatin nanoparticles are 150-220.

6. The nanomaterial of claim 1, encapsulating a drug or contrast agent.

7. A method for synthesizing ultrasmall gelatin nanoparticles, comprising:
conducting first desolvation to produce gelatin strands in a first solution and then setting the pH of the solution to charge and separate the strands;
adding tripolyphosphate (TPP) to the first solution to form gelatin-TPP-gelatin bridges;
conducting second desolvation of the gelatin-TPP-gelatin bridges in a second solution, wherein the second solution contains an TPP-Ethanol:Acetone mixture in a ratio between 1:1 and 1:5 and TPP between 0.005-0.025 vol % or a Glutaraldehyde-Ethanol:Acetone mixture in a ratio of glutaraldehyde-ethanol:Acetone range of 2.5-12% v/v to produce a colloid of self-assembled gelatin nanoparticles.

8. The method of claim 7, wherein the ethanol:acetone is between 1:1 and 1:5 volume.

9. The method of claim 8, wherein the ethanol:acetone is between 1:1 and 1:2 volume.

10. The method of claim 7, wherein the TPP is ˜0.01% volume.

11. The method of claim 7, comprising:
forming a gel nanoparticle solution from the second solution with a top solution having the ultrasmall nanoparticles and a bottom solution being a dense gel; and
collecting the ultrasmall nanoparticles from the gel nanoparticle solution.

12. The method of claim 11, wherein the forming comprises:
stirring the colloid;
cooling the colloid;
filtering the colloid via one or more less then 50 μm filters to form a filtered solution;
heating the filtered solution;
rapid cooling the filtered solution by mixing into water to form a gel nanoparticle solution;
allowing the gel nanoparticle solution to settle into a top solution with the ultrasmall nanoparticles and a bottom solution with a dense gel; and wherein the collecting comprises:
purifying the top solution via centrifugation to form fractional solutions; and
dialyzing a selected one of the fractional solutions to obtain an ultrasmall gelatin nanoparticle solution with gelatin nanoparticles sized less than 50 nm.

13. The method according to claim 12, wherein the dialyzing comprises sucrose-density centrifugation.

14. The method of claim 12, wherein the fractional solutions comprise a bottom 50%, and 20%, 10%, 5%, 2% fractional solutions, and the selected one of the fractional solutions is a top 2% fractional solution.

15. The method of claim 7, wherein a ratio of tripolyphosphate to the first solution is between 0.003-0.005% w/v (w/v: Ratio between amount of TPP (mg) and volume of acidified gelatin sol (mL)).

16. The method of claim 7, wherein the ratio of glutaraldehyde-ethanol:acetone of ˜12. % v/v.

17. The method of claim 7, comprising adding a drug or contrast agent to the first solution prior to or with the TPP.

18. The method of claim 7, comprising adding metal nanoparticles to the first solution prior to or with the TPP.

19. The method of claim 6, comprising additional steps adding a crosslinker mixture containing TPP, glutaraldehyde and PEG to colloid to form a nanocomposite of large gel nanoparticles decorated with satellite nanoparticles.
